(19) United States
(12) Patent Application Publication
(10) Pub. No.: US 2008/0184576 A1
(43) Pub. Date: Aug. 7, 2008

(54) Medical Treatment Aid For Use in Treating Children

(76) Inventors: Timothy Hodgetts, Birmingham (GB); Peter Francis Mahoney, Lincoln (GB); William Grant Phillip Eardley, Midlesbrough (GB); Linda Orr, Gloucester (GB); Christopher Russell Wright, Peterborough (GB)

Correspondence Address: JOHN S. PRATT, ESQ;KILPATRICK STOCKTON, LLP, 1100 PEACHTREE STREET, ATLANTA, GA, 30309, US

(21) Appl. No.: 11/914,395

(22) PCT Filed: May 11, 2006
(86) PCT No.: PCT/GB06/01747

§ 371 Date: Apr. 4, 2008

(30) Foreign Application Priority Data
May 14, 2005 (GB) 0509911.4

(51) Int. Cl. A61B 5/107

(52) U.S. Cl. 33/512

(57) ABSTRACT

A medical treatment aid is described which allows for the rapid classification of child patients in an emergency, casualty or trauma room environment, and further displays treatment information relevant to the classification of the patient in a readily accessible manner.

The present invention relates to apparatus for assisting in the treatment of children in a casualty or emergency room situation.

The resuscitation and other treatment of children who are seriously ill, traumatised or injured occasions significant anxiety for professional medical staff, both in ambulance services and hospital emergency departments. This is due to a relative unfamiliarity in dealing with seriously ill or injured children, such cases being comparatively less common than adult patients. The treatment of children thus necessitates rapid arithmetical calculations to determine the choice of equipment and/or dosages of drugs to be used for a particular age and/or weight of child. An added complication arises from the great range of size and weight of children. Drug dosages must be correct for a particular bodyweight, and treatment equipment such as needles, chest drains, breathing apparatus and so forth must be of the correct size to match the size of the child patient. Typically, therefore, in an emergency room situation with current art materials to hand, the child's weight is estimated by a simple calculation (such as child's age plus four, multiplied by two, giving a weight in kilograms) by a team leader. This of course is complicated if the child patient's age is unknown. The various calculations necessary to work out drug dosages and equipment sizes are then carried out by either the team leader or other team members.

This problem is global in scope and is particularly pertinent in circumstances where expert paediatric advice is not readily available (including the military field hospital, or treatment facilities of voluntary aid organisations). In an emergency situation, in which treatment must be rendered quickly and correctly, the possibility of and the consequences of error can put medical staff under great stress, which does not aid the process of determining correct drug dosages and/or correct sizes of equipment by calculation. Speed of assessment and thus intervention can play a crucial part in determining the outcome of an emergency situation.

What is required to solve this problem is a device which can aid the medical worker in rapidly and reliably assessing vital statistics of the child patient such as age, size and weight, and further assist in determining the correct dosages of common emergency room drugs and the correct sizes of common items of equipment which must be used.

Prior art in this field includes the ‘Broselow-Hinkle’ system, which is in effect a large tape-measure with which a child patient can be measured. This then results in a classification of the patient by colour-code, and the colour code relates to a bag of equipment. The tape also provides information on drug doses.

One of the disadvantages of this system is that the same tape with which one measures the child may well contain vital information which is necessary for treating the child—tape which may well become bloodied or otherwise soiled in use. Further, measurement of a body using a tape is complicated by the natural undulations of a body and may require more than one person to do the measurement, complicating the measuring process as the tape may become tangled or rucked up during the measurement.

Accordingly, the present invention provides a medical treatment aid, comprising a flexible sheet provided with a central panel having gradations upon which patients can be laid, which gradations enable the patient to be quickly classified by size and one or more peripheral areas which present charts or tables or diagrams relating to dosages of drugs and/or sizes of equipment to be used on the patient according to that classification.

This invention makes use of the well-established medical principle that between the ages of 1 to 10, the overall length of a child is proportional to both age and weight. As drug dosages for children are calculated on patient weight, this invention provides a ready means of establishing drug dosages as well as sizes of critical equipment to be used without the necessity for stressful and time-pressured calculation.

Advantages of the current invention are: the provision of graphical and textual support to each individual member of an emergency team, which facilitates ‘horizontal’ care (where many people perform various tasks at the same time under the direction of a team leader, as opposed to ‘vertical’ care where tasks occur one after the other); the provision of guidance to an emergency team leader which enables consistent treatment within different geographical locations and despite different care providers; the current invention is unique in providing graphical information to assist in the rapid sizing of critical equipment, speeding preparation for critical medical intervention.

Also, the invention makes measurement of a patient's length easy; once the sheet is spread out upon an emergency bed or other flat surface, the patient's length can easily be read from the gradations on the central panel. Further, the peripheral areas of the invention upon which treatment information is displayed are less liable to be soiled than the information on the prior art tape.

In a preferred feature of the invention, equipment sizes are indicated by means of pictorial ‘silhouettes’ of the various equipment items, such that equipment of the appropriate size for the classification of patient can be selected by means of quick ‘matching’ of the equipment against the appropriate silhouette.

In a preferred embodiment of the invention, the gradations on the central panel show correspondence of patient length with a range of particularly easily identifiable and intuitive symbols, such as animals, where the smallest gradation, for example, corresponding to a child of up to 1 year of age, is a mouse, the next gradation being a rabbit, the next a cat and so on until the final gradation is represented by an animal such as an elephant.

Typically, for patients in the age range 0 to 12, it is advantageous to group them into 7 or 8 bands or classifications of size, such as, for particular ease of memory and recognition, grading by animal sizes where the animals selected for the various sizes are such as to intuitively reflect the size classification of the patient. By way of example, those size classifications, with the smallest patient gradation first, would be as follows: mouse, rabbit, cat, dog, kangaroo, lion, camel, elephant.

In this embodiment, the peripheral areas which present charts will show information related to the symbol. So, for example, a peripheral area having a chart of drug dosages will show a particular drug dosage for a patient identified as a ‘mouse’, and a separate, possibly different particular drug dosage for a patient identified as a ‘cat’, and so on. Such classification of patient size reduces the possibility of error during reference to the information on the peripheral areas of the invention.

In another embodiment of the invention, the central panel comprises a suitable weight-sensing device which gives an actual measurement of weight, thus removing uncertainty about patient weight entirely and allowing drug dosages to be calculated much more finely, or giving quicker and clearer information allowing patient weight classification in an even shorter time scale.

In this embodiment, the invention would be in the form of a flexible sheet, where the central panel may consist of a multilayer composite containing either one or a number of sensing devices for measuring the weight of the patient. Alternatively the central panel may comprise one or more inflatable sections, and a sensor or sensors capable of measuring air pressure as a result of load placed upon the central panel and producing a weight measurement from that information.

The weight-sensing device could advantageously give a reading of patient weight by gradated classification, such as the easily identifiable symbols noted earlier such as animals.

Further, the invention could comprise a computer which has as its input the actual measurement of weight, from which dosages of various commonly used emergency drugs are calculated and then displayed to the medical team.

The invention will now be described further, by way of example only, by means of reference to the attached drawings.

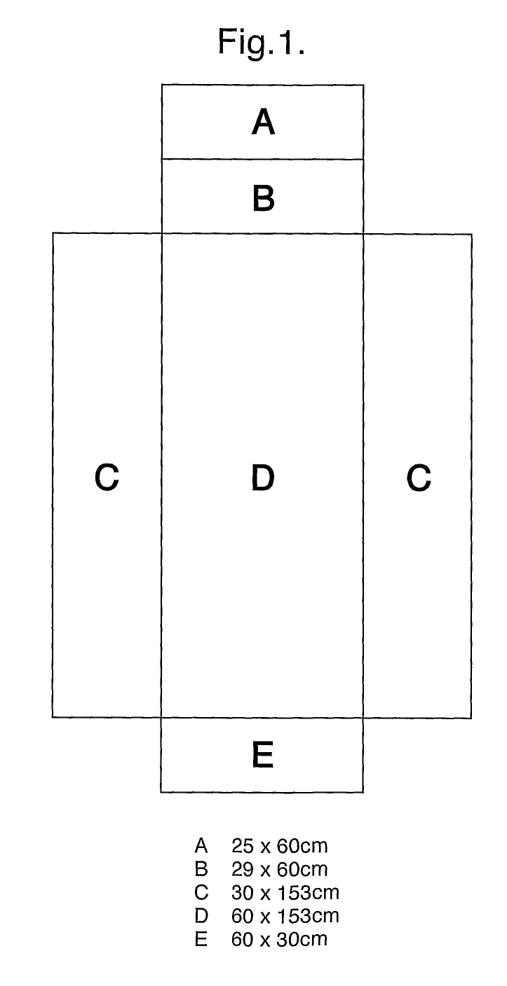

FIG. 1 shows a general layout of the invention. The central panel, D, is the area upon which the patient is laid. Peripheral areas A, B, C, and E are all available for the presentation of information pertinent to patient treatment.

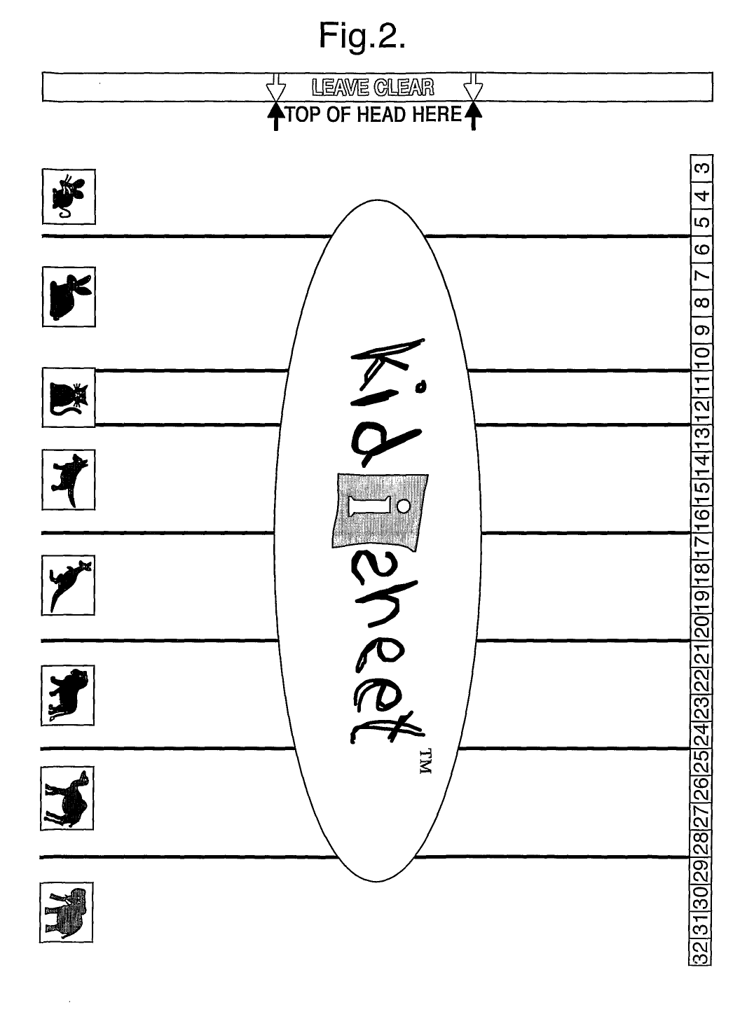

FIG. 2 shows a representative layout of the central panel of the invention.

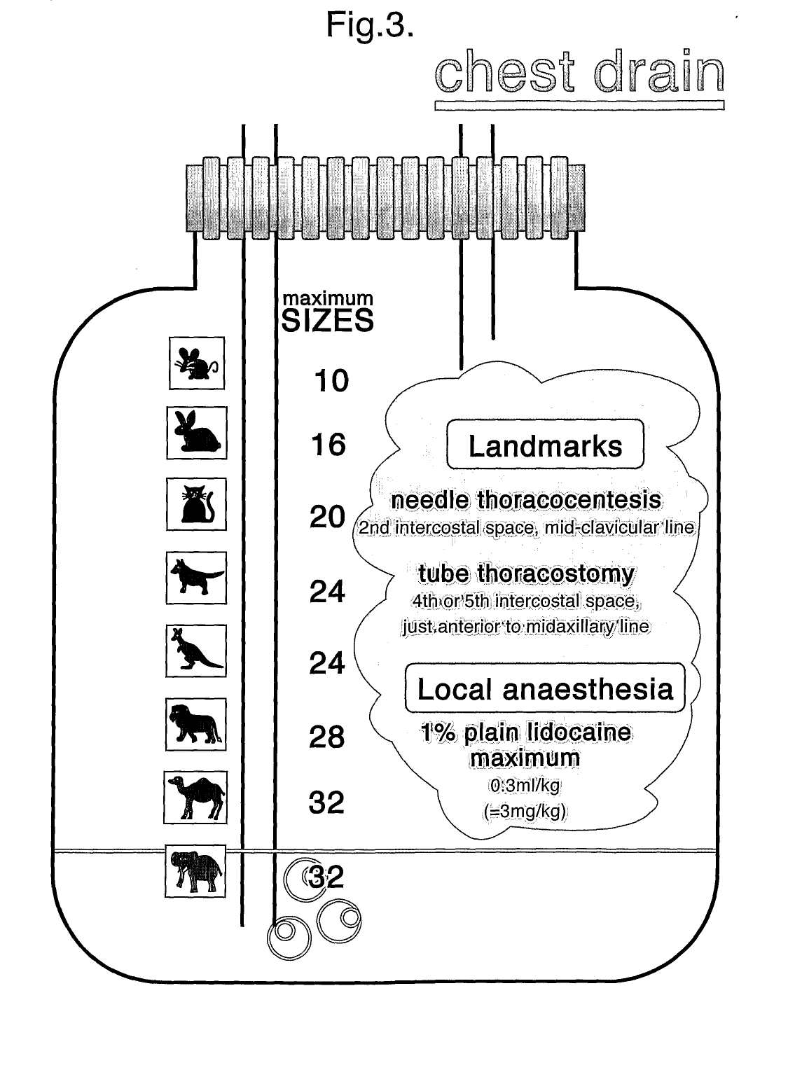

FIG. 3 shows a chart such as would be present on one of the peripheral areas of the invention, giving information on chest drain equipment in accordance with a patient classification system.

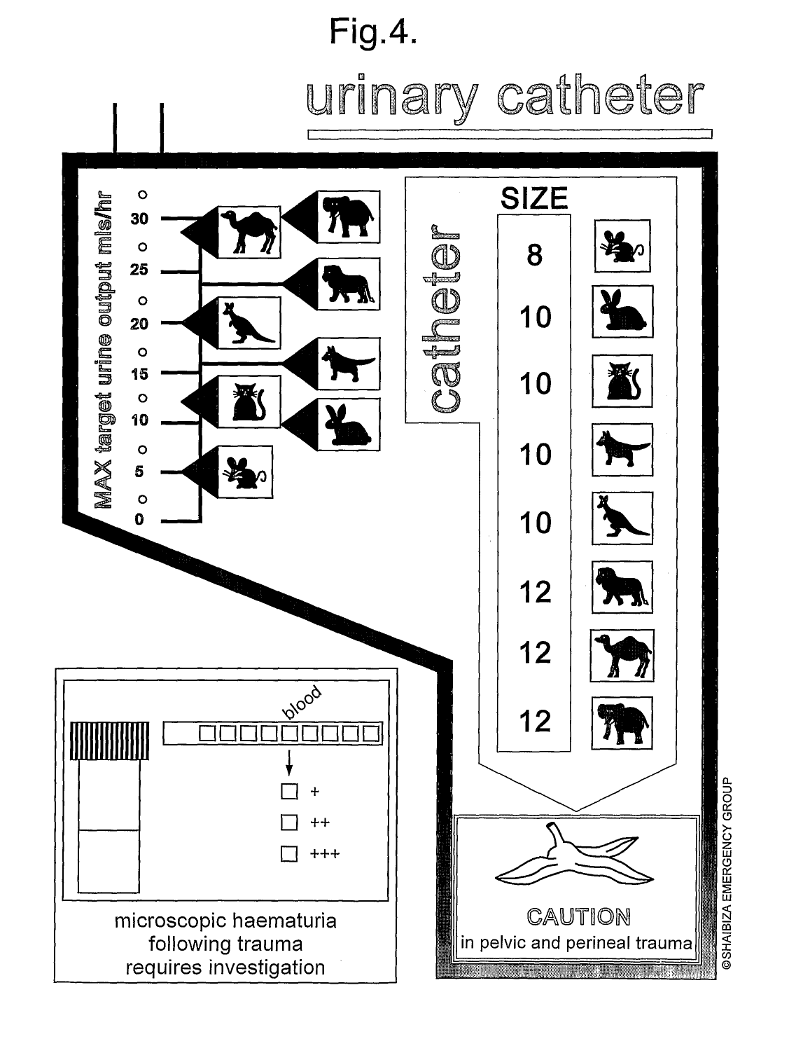

FIG. 4 shows a chart such as would be present on one of the peripheral areas of the invention, giving information on urinary catheter equipment in accordance with a patient classification system.

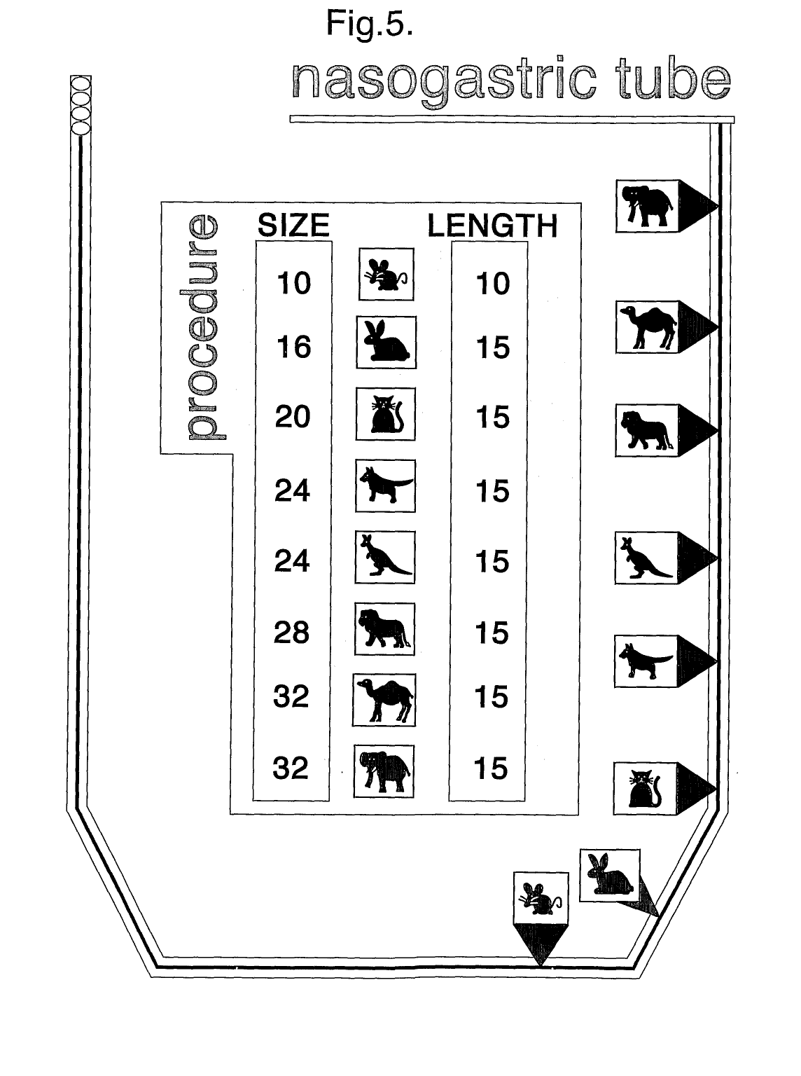

FIG. 5 shows a chart such as would be present on one of the peripheral areas of the invention, giving information on nasogastric tube equipment sizing in accordance with a patient classification system.

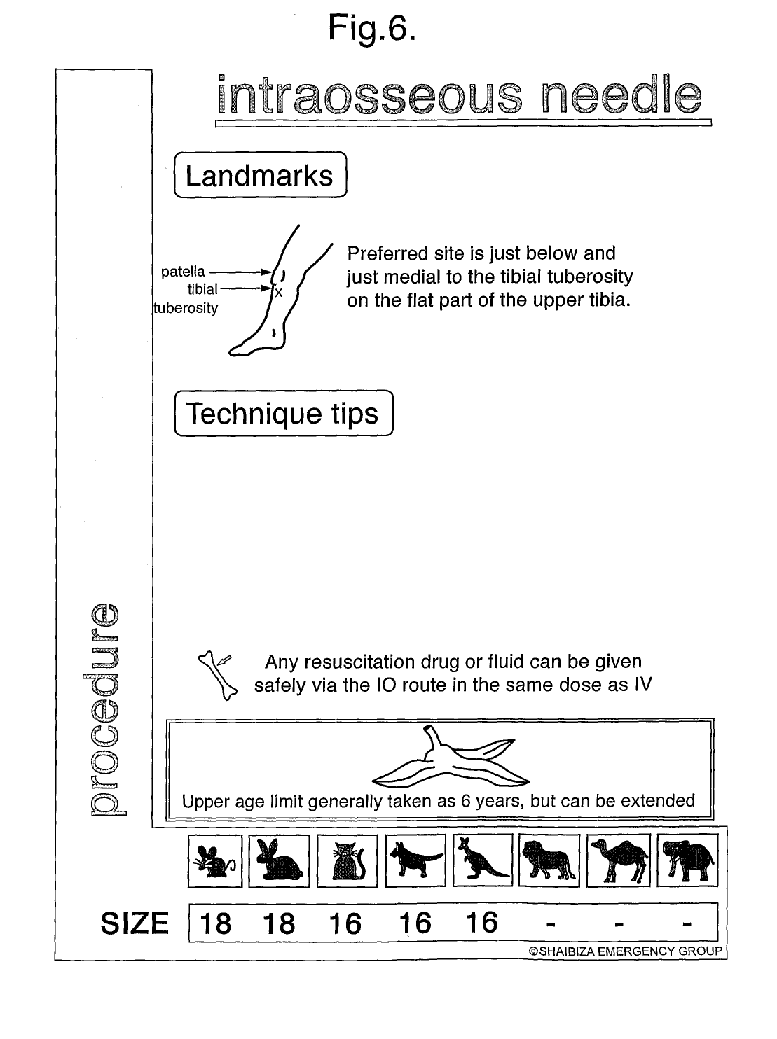

FIG. 6 shows a chart such as would be present on one of the peripheral areas of the invention, giving information on intraosseous equipment sizing in accordance with a patient classification system.

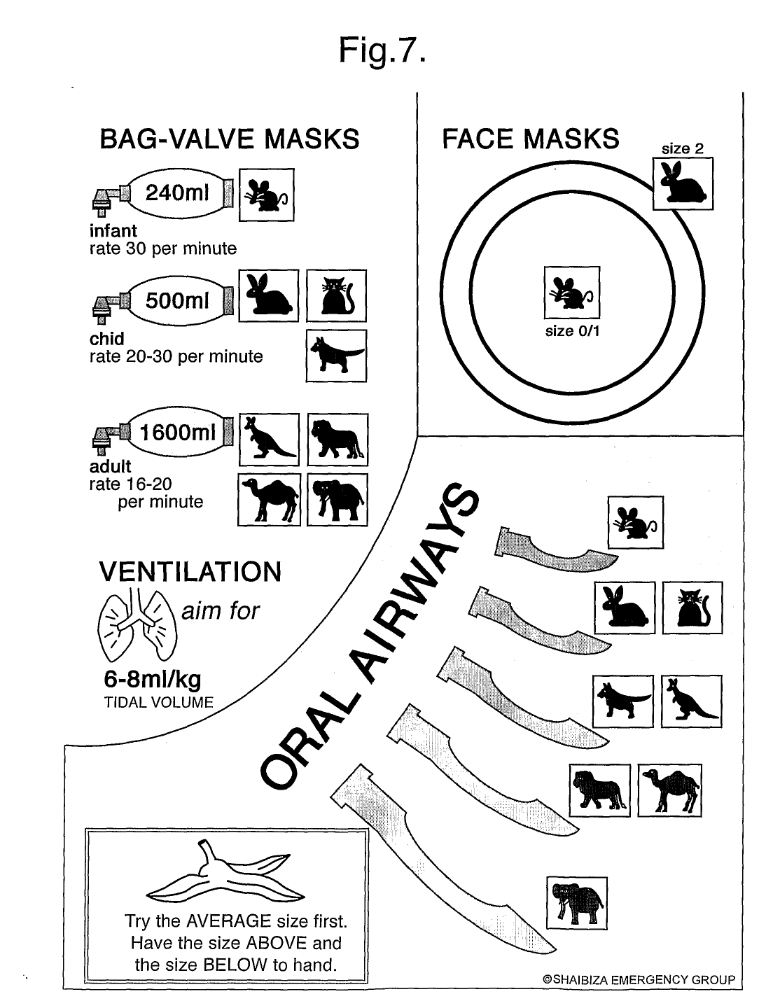

FIG. 7 shows a chart such as would be present on one of the peripheral areas of the invention, giving information on breathing equipment sizing in accordance with a patient classification system.

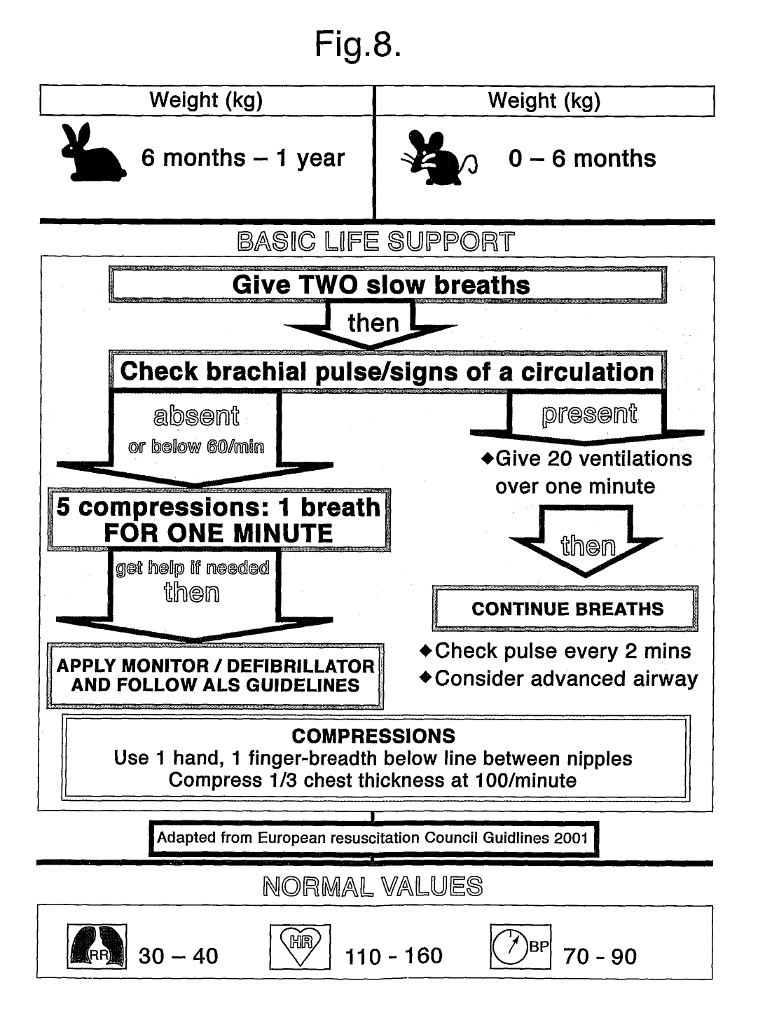

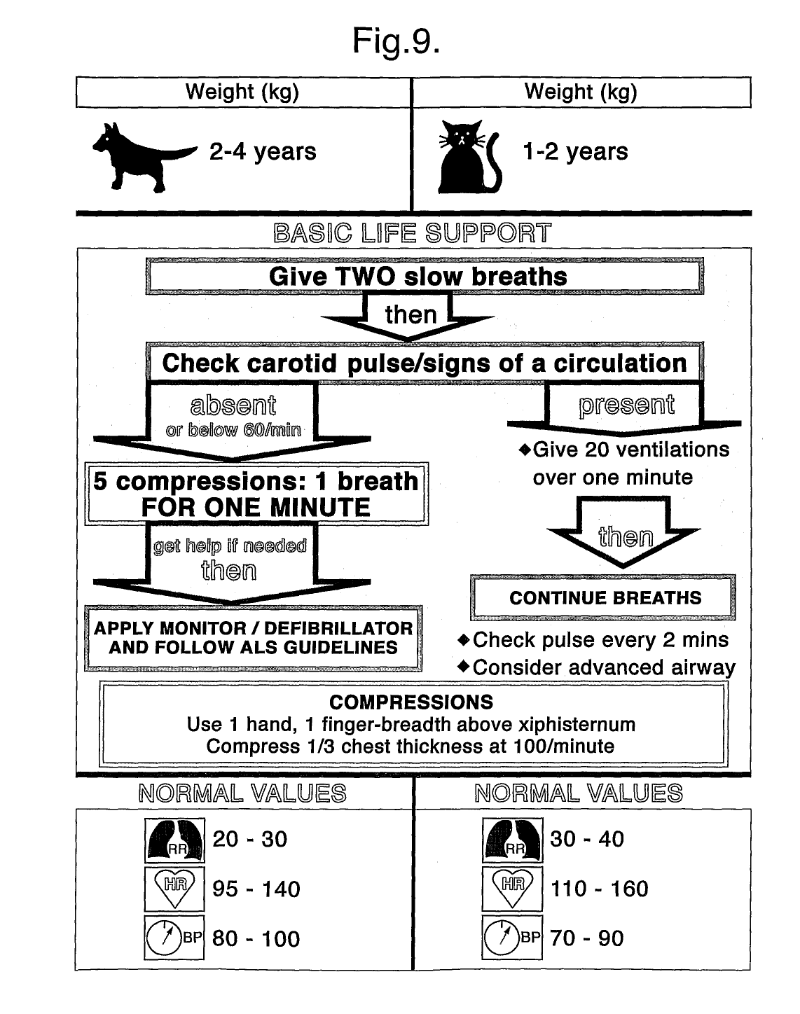

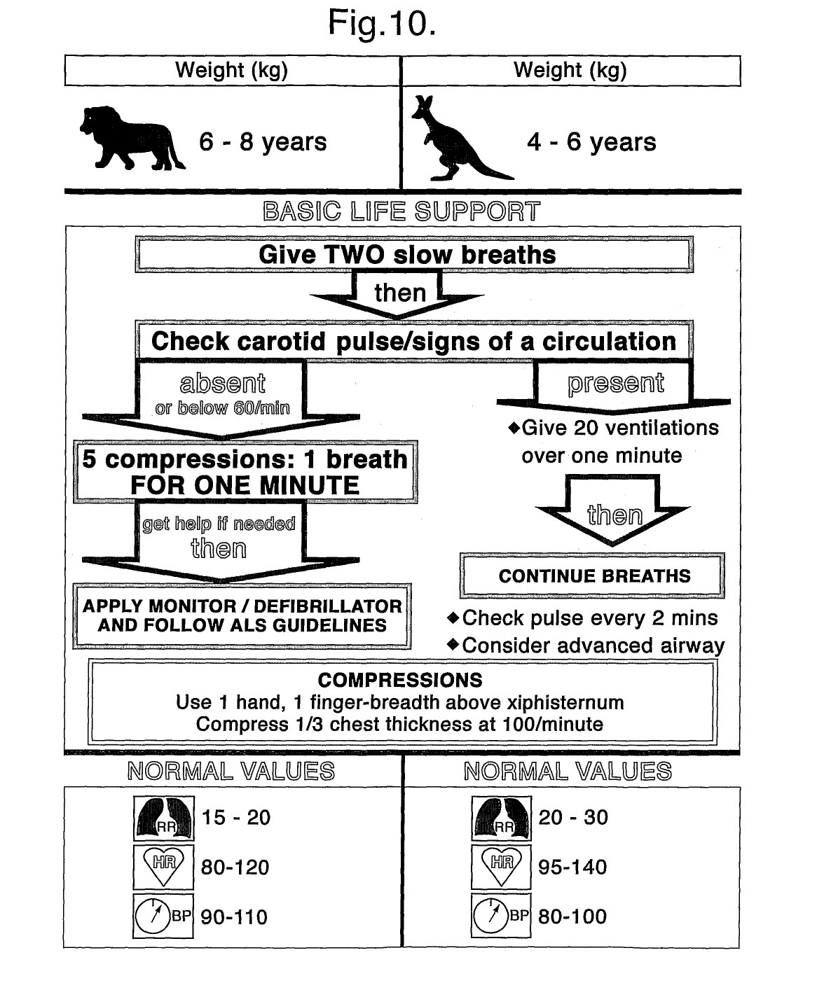

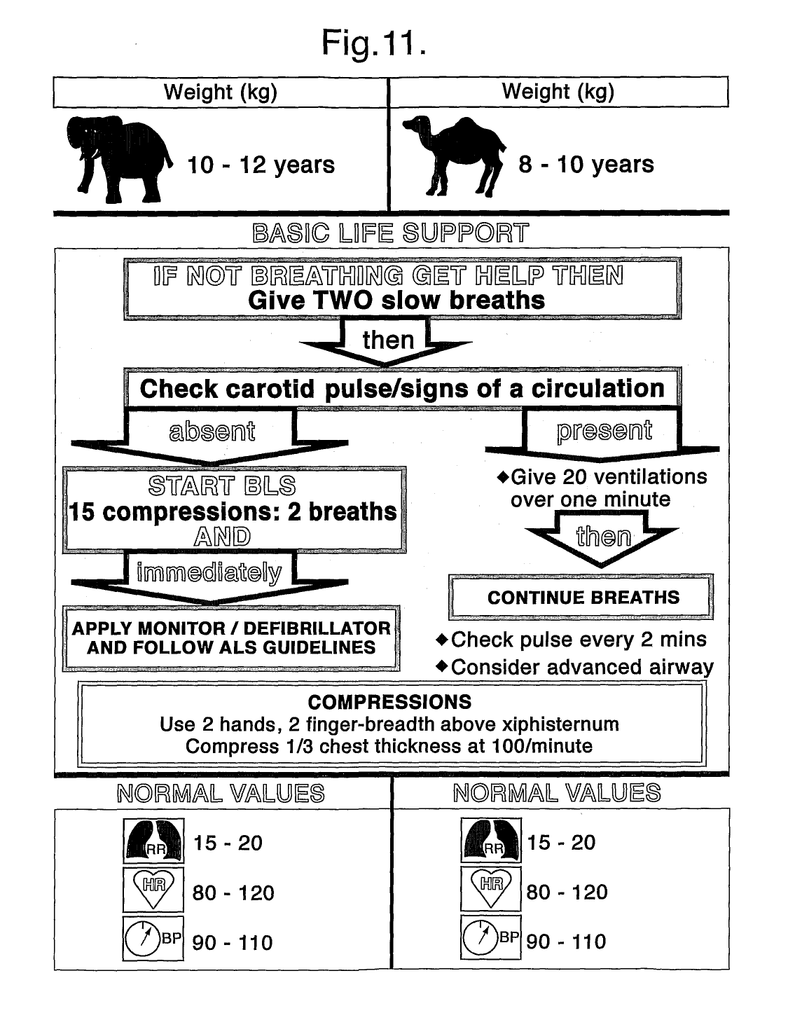

FIGS. 8 to 11 show a table such as would be present on one of the peripheral areas of the invention, giving information on patient resuscitation in accordance with a patient classification system.

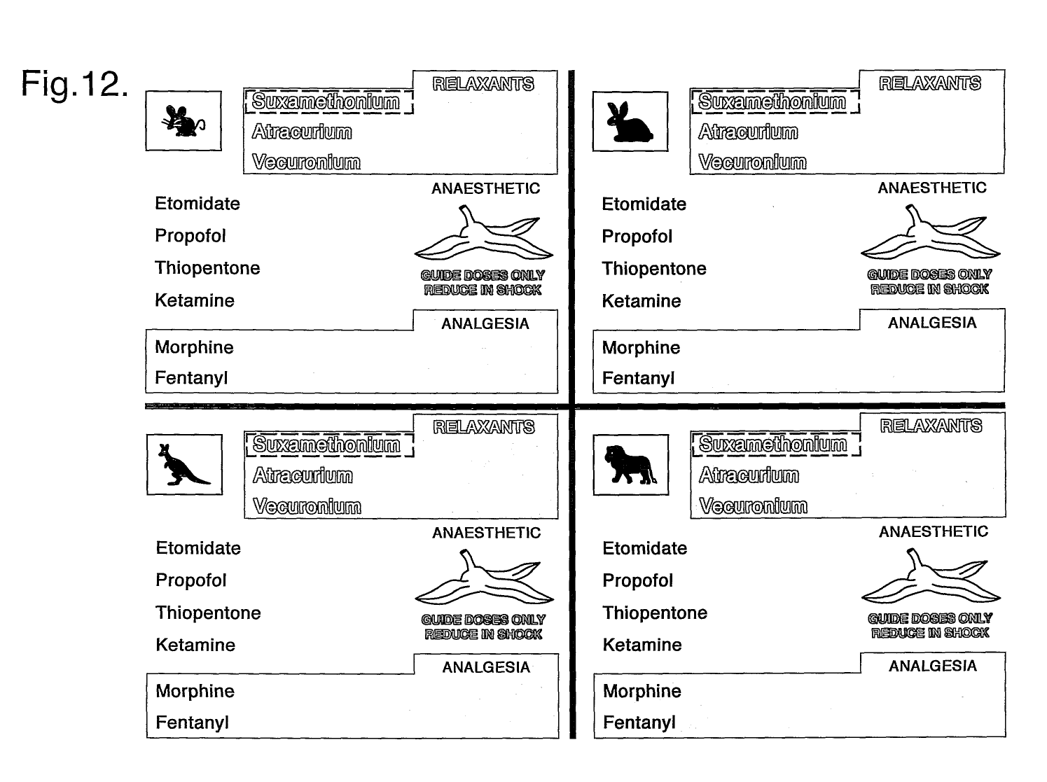

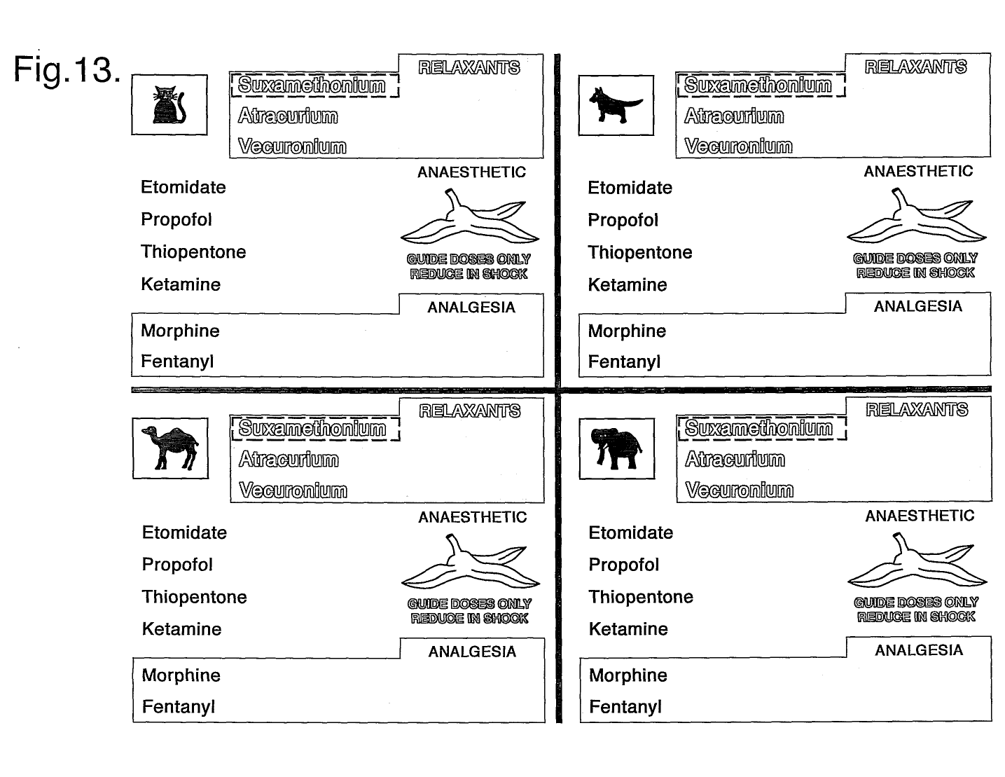

FIGS. 12 and 13 show a table such as would be present on one of the peripheral areas of the invention, giving information on drug dosages in accordance with a patient classification system.

FIG. 1 shows a general layout of the invention, with a central panel and peripheral areas. It comprises a plastic, permeable or impermeable paper, or other material sheet which can be laid upon an emergency bed. Whilst almost any flexible material can be used for the invention, it would preferably be impermeable so that, for example, when laid upon a bed in a hospital it would prevent the transmission of fluids through to the bed below, thus helping to prevent contamination of the bed which could effect future patients.

FIG. 2 shows a typical layout of the central panel of the invention, upon which are various gradations which correspond to certain easily identifiable symbols, in this case animals.

FIGS. 3 to 13 are indicative of the types of information presented upon the peripheral areas of the invention. Typically, these will be presented at points around the periphery of the central panel which correspond most clearly with the emergency team member likely to be present at that position. For example, information about anaesthetics (FIGS. 12 and 13) appears on a section of the periphery of the invention at the head of the patient where the anaesthetist would typically be located.

FIG. 7 shows a typical presentation of information on equipment sizes in the form of silhouettes, which enable quick selection of equipment size appropriate for the classification of patient.

1. A medical treatment aid comprising a flexible sheet provided with a central panel having gradations upon which patients can be laid, which gradations immediately enable the patient to be classified by size and one or more peripheral areas which present charts or tables or diagrams relating to dosages of drugs and/or sizes of equipment to be used on the patient according to that classification.

2. A medical treatment aid as claimed in claim 1, wherein the gradations on the central panel show correspondence of patient length with a range of particularly easily identifiable symbols.

3. A medical treatment aid as claimed in claim 2, wherein the particularly easily identifiable symbols are pictorial representations of animals.

4. A medical treatment aid as claimed in claim 1 wherein the peripheral areas show information regarding sizes of equipment to be used in the form of silhouettes.

5. A medical treatment aid comprising a flexible sheet provided with a central panel upon which patients can be laid and one or more peripheral areas which present charts relating to dosages of drugs and/or sizes of equipment to be used on the patient depending on weight, further comprising a weight-sensing means in the central panel which gives an actual measurement of weight of any patient placed upon it.

6. A medical treatment aid as claimed in claim 5, wherein the weight-sensing means displays the weight of the patient in the form of one of several easily identifiable gradated classifications.

7. A medical treatment aid as claimed in claim 5, further comprising a computing device which rapidly calculates selected drug dosages automatically in response to the weight of the patient upon the central panel.
